(19) United States
(12) Patent Application Publication
(10) Pub. No.: US 2003/0086951 A9
(43) Pub. Date: May 8, 2003

(54) Cosmetic composition comprising at least one fiber and at least one wax

(76) Inventors: Bertrand Piot, Paris (FR); Nathalie Collin, Sceaux (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 09/984,184

(22) Filed: Oct. 29, 2001

(30) Foreign Application Priority Data
Oct. 27, 2000 (FR) 00 13866

(51) Int. Cl. A61K 7/021; A61K 7/00; A61K 7/06

(52) U.S. Cl. 424/401; 424/70.1

(57) ABSTRACT

A composition comprising, in a physiologically acceptable medium comprising at least one aqueous phase, at least one fiber and an aqueous microdispersion of particles of at least one wax. The invention also relates to a care and make-up process for keratinous materials.

The invention makes it possible to obtain an adherent deposit of the at least one fiber on the keratinous materials.

The present invention relates to a composition comprising, in a physiologically acceptable medium, at least one fiber and at least one wax, intended, for example, for the cosmetic field. The invention also relates to a method for make-up or cosmetic care of a keratinous material comprising applying, to the keratinous material, a cosmetic composition comprising, in a physiologically acceptable medium, at least one fiber and at least one wax. The composition and the method according to the invention are intended, for example, for keratinous materials such as skin (including lips), and skin appendages such as eyelashes, eyebrows, hair and nails. In one embodiment of the invention, the keratinous material is of human origin. In one embodiment, the invention relates to a mascara.

The composition according to the invention may be provided in the form chosen, for example, from a covering for eyelashes, such as a mascara, an eyeliner, a product for lips, a blusher, an eyeshadow, a foundation, a make-up product for the body, a concealer, a nail varnish and a skin-care product.

Make-up products are commonly used to provide color, to enhance certain parts of the skin or skin appendages, to give a shiny appearance to skin or skin appendages, to give a matte appearance to skin or skin appendages, and to give a satiny appearance to skin or skin appendages. These products may be usually applied in the form of a thin, uniform layer.

It is known from the state of the art that fibers may be used in compositions for make-up or care of keratinous materials in order to enhance their cosmetic properties.

For the application of make-up to eyelashes, the document JP-A-3-153613, for example, teaches the use of fibers in mascara compositions for conferring a stretching and thickening effect on the eyelashes. The documents JP-A-6-9340 and JP-A-7-179323, for example, describe mascara compositions comprising fibers and polymers in an aqueous dispersion.

For the application of make-up to skin, the document JP-A-7-196440, for example, discloses use of fibers to confer a velvety feel on the skin.

However, during the application of these compositions to the keratinous materials, it may be difficult for the fibers to adhere to the keratinous materials. The user therefore may need to apply the composition to the keratinous materials several times in order to deposit a sufficient quantity of fibers to obtain the desired cosmetic properties. This procedure may require devoting a certain amount of time to the application of make-up in order to obtain the desired results for the make-up application. However, this time may be perceived as being too long by users pressed for time. Therefore, there is a need to have available a composition containing fibers which make it possible to rapidly and easily obtain the desired make-up application.

Moreover, fibers which do not adhere to keratinous materials may therefore tend to become detached from their support and be eliminated over time. The elimination of these fibers may cause a perceptible reduction in the desired cosmetic properties provided by the fibers, thus requiring re-application of the product. Furthermore, in a mascara, for example, the fibers, on becoming detached from the eyelashes, can become lodged in the eye and may cause eye discomfort.

One embodiment of the present invention is to make available a cosmetic composition comprising fibers which adhere well to keratinous materials.

The inventors have surprisingly found that the use of a microdispersion of at least one wax in a composition comprising at least one fiber may make it possible to obtain a composition having at least one of the following properties: may be easily applied to keratinous materials and may allow the fibers to be rapidly deposited on the keratinous materials. In addition, the composition applied to the keratinous materials may dry rapidly, thus promoting a rapid and adhering retention of fibers on the keratinous materials. Also, when the composition is a mascara, the make-up obtained may have at least one of the following properties: rapid thickening of the eyelashes and lengthening of the eyelashes. Instant loading of the eyelashes may thus be observed.

One embodiment of the invention is a composition comprising, in a physiologically acceptable medium comprising at least one aqueous phase, at least one fiber and an aqueous microdispersion of particles of at least one wax.

Another embodiment of the invention is a mascara comprising, in a physiologically acceptable medium comprising at least one aqueous phase, at least one fiber and an aqueous microdispersion of particles of at least one wax.

Yet another subject of the invention is a mascara product comprising:

a reservoir containing a mascara composition comprising, in a physiologically acceptable medium comprising at least one aqueous phase, at least one fiber and an aqueous microdispersion of particles of at least one wax; and

a system for applying the mascara composition onto a keratinous material, such as for example, eyelashes.

Another embodiment of the invention is a cosmetic method for make-up or care of a keratinous material comprising applying to the keratinous material a composition comprising, in a physiologically acceptable medium comprising at least one aqueous phase, at least one fiber and an aqueous microdispersion of particles of at least one wax.

In one embodiment, the invention may be a method for covering eyelashes comprising applying to the eyelashes a composition comprising, in a physiologically acceptable medium comprising at least one aqueous phase, at least one fiber and an aqueous microdispersion of particles of at least one wax.

Another embodiment of the invention may be a method for obtaining a deposit adhering to a keratinous material comprising applying to the keratinous material a composition comprising, in a physiologically acceptable medium comprising at least one aqueous phase, at least one fiber and at least one wax to obtain the deposit which adheres to the keratinous materials.

Yet another embodiment of the invention may be a method for obtaining a deposit adhering to a keratinous material comprising applying to the keratinous material a composition comprising, in a physiologically acceptable medium comprising at least one aqueous phase, at least one fiber and an aqueous microdispersion of particles of at least one wax to obtain the deposit which adheres to the keratinous material.

In the present invention, the expression “physiologically acceptable medium” is understood to mean a medium compatible with the keratinous materials, for example, a cosmetic medium.

The aqueous phase of the composition may comprise water and may also comprise a mixture of water and at least one water-miscible solvent chosen, for example, from lower monoalcohols having from 1 to 5 carbon atoms such as, for example, ethanol, isopropanol, glycols having from 2 to 8 carbon atoms such as those chosen, for example, from propylene glycol, ethylene glycol, 1,3-butylene glycol, and dipropylene glycol, (C₃-C₄) ketones, and (C₂-C₄)aldehydes. The aqueous phase (water and optionally at least one water-miscible organic solvent) may be present in the composition according to the invention in an amount ranging, for example, from 5% to 99.4% by weight relative to the total weight of the composition.

A. The at Least One Fiber

For purposes of the invention, the expression “at least one fiber” is be understood to mean an object having a length, L, and a diameter, D, such that L is greater than D, wherein D is the diameter of the circle in which the section of the fiber is inscribed. In one embodiment of the invention, the ratio of L to D, (the L/D ratio or shape factor) may range, for example, from 3.5 to 2500, such as from 5 to 500, and further still from 5 to 150.

Non-limiting examples of the at least one fiber which may be used in the composition of the invention may be chosen from inorganic and organic fibers of synthetic and natural origin. The fibers may be those chosen, for example, from short and long, unitary and organized, fibers, such as for example, plaited, hollow and solid fibers. The fiber shape may be of any type, for example, fiber shapes chosen from circular and polygonal, such as, square, hexagonal and octagonal cross-sections according to the specific application envisaged. In one embodiment of the invention, the fibers' ends may be blunt or smooth to avoid hurting oneself.

In another embodiment, the fibers may have a length ranging, for example, from 1 μm to 10 mm, such as, from 0.1 mm to 5 mm and further still, from 1 mm to 3.5 mm. In yet another embodiment, the fibers' cross-section may be contained within a circle having a mean diameter ranging, for example, from 2 nm to 500 μm, such as, from 100 nm to 100 μm and further still from 1 μm to 50 μm.

The weight or titre of the fibers is often given in denier or decitex. A denier indicates the weight in grams of 9000 meters of yarn. A decitex (dtex) is the weight in grams of 10,000 meters of yarn. In one embodiment of the invention, the fibers have a titre ranging, for example, from 0.15 to 30 denier, such as from 0.18 to 18 denier.

Non-limiting representatives of fibers may be those used in the manufacture of textiles such as fibers chosen, for example, from silk fibers; cotton fibers; wool fibers; flax fibers; cellulose fibers, such as celluloses extracted from wood, plants, and algae; rayon fibers; polyamide fibers (NYLON); viscose fibers; acetate fibers, such as rayon acetate, poly(p-phenylene-terephthalamide) (or aramide) fibers, such as KEVLAR; acrylic polymer fibers, such as for example, polymethyl methacrylate and poly-2-hydroxyethyl methacrylate; polyolefin fibers; polyethylene fibers; polypropylene fibers; glass fibers; silica fibers; carbon fibers, such as, for example, graphite; polytetrafluoroethylene fibers, such as TEFLON; insoluble collagen fibers; polyester fibers; polyvinyl chloride fibers; polyvinylidene chloride fibers; polyvinyl alcohol fibers; polyacrylonitrile fibers; chitosan fibers; polyurethane fibers; polyethylene phthalate fibers; and fibers formed from mixtures of polymers such as those mentioned above, for example, polyamide/polyester fibers.

Further non-limiting examples of fibers which may be used in accordance with the invention, are those chosen, for example, from surgical fibers such as resorbable synthetic fibers prepared from glycolic acid and caprolactone (“MONOCRYL” from Johnson & Johnson); resorbable synthetic fibers of lactic acid and glycolic acid copolymer type (“VICRYL” from Johnson & Johnson); polyterephthalic ester fibers (“ETHIBOND” from Johnson & Johnson); and stainless steel wires (“ACIER” from Johnson & Johnson). In one embodiment, the stainless steel wires may be in a composition of the invention used as nail varnish.

Moreover, the fibers which may be used in accordance with the invention, may optionally be processed by a least one method chosen from surface-treatment and coating. Non-limiting representatives of coated fibers may be those chosen, for example, from polyamide fibers coated with copper sulphide for an antistatic effect (for example “R-STAT” from Rhodia); fibers coated with another polymer allowing a particular organization of the fibers (specific surface-treatment); and fibers with a surface-treatment which induces color/hologram effects (for example, “LUREX” fiber from Sildorex).

In one embodiment of the invention, the fibers may be chosen from fibers of synthetic origin, such as organic fibers, for example, surgical fibers. In another embodiment, water-insoluble fibers may be used.

In yet another embodiment, fibers which can be used in the composition according to the invention may be chosen, for example, from polyamide fibers, cellulose fibers, poly-p-phenylene terephthalamide fibers, and polyethylene fibers. The fiber length, (L), may range, for example, from 0.1 mm to 5 mm, such as from 0.25 mm to 1.6 mm. In addition the fiber mean diameter may range, for example, from 1 μm to 50 μm.

In one embodiment, it is possible to use the polyamide fibers marketed by Etablissements P. Bonte under the name “POLYAMIDE 0.9 Dtex 3 mm” having a mean diameter of 6 μm, a titre of 0.9 dtex and a length ranging, for example, from 0.3 mm to 5 mm. Other non-limiting representative fibers which may be used are chosen, for example, from poly-p-phenylene terephthalamide fibers having a mean diameter of 12 μm and a length of 1.5 mm such as those sold under the name “KEVLAR FLOC” by the company DuPont Fibers, cellulose (or rayon) fibers having a mean diameter of 50 μm and a length ranging from 0.5 mm to 6 mm such as those sold under the name “NATURAL RAYON FLOCK FIBER RC1BE-N003-M04” by the company Claremont Flock, and polyethylene fibers such as those sold under the name “SHURT STUFF 13 099 F” by the company Mini Fibers.

The fibers may be present in the composition according to the invention in an amount ranging, for example, from 0.01% to 10% by weight relative to the total weight of the composition, such as from 0.1% to 5% by weight, and further from 0.3% to 2% by weight relative to the total weight of the composition.

B. The Microdispersion of at Least One Wax

The composition according to the invention also comprises, moreover, an aqueous microdispersion of particles of at least one wax. For the purposes of this invention, the expression “an aqueous microdispersion of at least one wax” is understood to mean an aqueous dispersion of particles of at least one wax wherein the mean particle size of the at least one wax may be, for example, less than or equal to 1 μm.

In the present invention, at least one wax may be understood to mean a lipohilic compound which is solid at room temperature (25° C.), and may undergo a reversible change from solid state to liquid state. The at least one wax may have a melting point of at least 30° C., for example, up to 120° C. By bringing the at least one wax to the liquid state (melting), it is possible to make it miscible with oils and to form a microscopically homogeneous mixture. By bringing the temperature of the mixture back to room temperature, recrystallization of the at least one wax in the oils of the mixture is obtained.

The melting point of the at least one wax may be measured using a Differential Scanning Calorimeter (DSC), for example, the calorimeter sold under the name DSC 30 by the company Metler. One technique for determining the melting point of the at least one wax is by placing a 15 mg sample of product in a crucible and subjecting the sample to a first rise in temperature ranging, for example, from 0° C. to 120° C., at a heating rate of 10° C./minute. The sample is then cooled from 120° C. to 0° C. at a cooling rate of 10° C./minute and finally, the sample is subjected to a second temperature rise ranging, for example, from 0° C. to 120° C. at a heating rate of 5° C./minute. During the second rise in temperature, the variation of the difference in the power absorbed by the empty crucible and by the crucible containing the sample of product is measured as a function of the temperature. The melting point of the compound is the value of the temperature corresponding to the summit of the peak of the curve representing the variation of the difference in power absorbed as a function of the temperature.

The at least one wax microdispersions are stable dispersions of colloidal particles of wax, and are, for example, described in “Microemulsions Theory and Practice”, L. M. Prince Ed., Academic Press (1977), pages 21-32, incorporated by reference herein.

For example, these at least one wax microdispersions may be obtained by melting wax in the presence of at least one surfactant, optionally together with a portion of water, and then gradually adding hot water with stirring. The intermediate formation of a water-in-oil type emulsion is observed followed by a phase inversion with final production of an oil-in-water type microemulsion. On cooling, a microdispersion of solid colloidal particles of at least one wax may be obtained. The wax microdispersions may also be obtained by stirring the mixture of at least one wax, at least one surfactant, and water using stirring means such as for example, ultrasound, high-pressure homogenizers and turbines.

The particles of at least one wax in the form of a microdispersion may have, for example, a mean particle size of less than 1 μm (ranging, for example, from 0.02 μm to 1 μm), such as, less than 0.5 μm (for example, ranging from 0.051 μm to 0.5 μm).

These particles may comprise one wax or a mixture of waxes. In addition, these particles may further comprise a minor proportion of at least one compound chosen, for example, from oily fatty additives, pasty fatty additives, oily and pasty fatty additives, surfactants, customary fat-soluble additive/active agents.

Non-limiting examples of at least one wax which may be used in the compositions according to the invention may be waxes which are solid and rigid at room temperature, chosen, for example, from waxes of animal origin, waxes of plant origin, waxes of mineral origin, and waxes of synthetic origin. In one embodiment, the at least one wax employed in the composition may have a melting point greater than 45° C., for example, greater than 55° C. The wax may also have a hardness ranging, for example, from 0.05 MPa to 15 MPa, such as from 6 MPa to 15 MPa. The hardness is determined by measuring the compacting force measured at 20° C. using a texturometer sold under the name TA-TX2i by the company RHEO, equipped with a stainless steel cylinder having a diameter of 2 mm, moving at the measuring speed of 0.1 mm/s and penetrating into the wax at a penetration depth of 0.3 mm. To carry out the measurement of hardness, the wax is melted at a temperature equal to the melting point of the wax +20° C. The molten wax is poured into a container having a diameter of 30 mm and a depth of 20 mm. The wax is recrystallized at room temperature (25° C.) for 24 hours, and then the wax is stored for at least 1 hour at 20° C. before carrying out the measurement of hardness. The value of the hardness is the measured compacting force divided by the surface of the texturometer cylinder in contact with the wax.

Non-limiting representatives of at least one wax may be chosen, for example, from hydrocarbon waxes such as those chosen from beeswax, lanolin wax, Chinese waxes, rice wax, Carnauba wax, candelilla wax, ouricury wax, Esparto wax, cork fiber wax, sugarcane wax, Japan wax, sumac wax, montan wax, microcrystalline waxes, paraffins, ozokerite; polyethylene waxes, waxes obtained by Fisher-Tropsch synthesis, waxy copolymers, and esters of waxes.

Other non-limiting representatives of at least one wax may include those waxes obtained by catalytic hydrogenation of oils chosen, for example, from animal oils with linear or branched (C₈-C₃₂) fatty chains and vegetable oils with linear or branched (C₈-C₃₂) fatty chains. Among these, there may be mentioned, for example, those chosen from hydrogenated jojoba oil, hydrogenated sunflower oil, hydrogenated castor oil, hydrogenated copra oil and hydrogenated lanolin oil.

Silicone waxes and fluorinated waxes may also be mentioned as examples of at least one wax.

It may also be possible to use commercial mixtures of self-emulsifying waxes containing a wax and surfactants. Non-limiting representatives of these commercial mixtures, for example, are chosen from the wax marketed under the name ‘CIRE AUTO LUSTRANTE OFR’ by Tiscco, which contains Carnauba and paraffin waxes in combination with nonionic surfactants, and the self-emulsifiable wax marketed under the name ‘CERAX A.O. 28/B’ by Ceresine, which contains Esparto wax in combination with a nonionic surfactant. These commercial mixtures may make it possible to prepare microdispersions of waxes by simply adding water.

Other non-limiting examples of commercial wax mixtures which may be used in accordance with the invention, may be chosen from the products, ‘AQUACER’ by Byk Cera, such as a mixture of synthetic and natural waxes with an anionic emulsifier (AQUACER 520), polyethylene wax with a nonionic emulsifier (AQUACER 514 or 513), and polymeric wax with an anionic emulsifier (AQUACER 511). In addition, there may also be mentioned the mixture of polyethylene and paraffin waxes with a nonionic emulsifier, ‘JONWAX 120’ from Johnson Polymer.

The wax may be present in the composition according to the invention in a dry matter content ranging, for example, from 0.1% to 50% by weight relative to the total weight of the composition, such as, from 0.5% to 30% by weight, and further, from 1% to 20% by weight relative to the total weight of the composition.

The composition in accordance with the invention, may further comprise an effective quantity of at least one surfactant which may make it possible to obtain a wax microdispersion or a stable, final composition. In one embodiment, the at least one surfactant may be present in the composition in accordance with the invention, in an amount ranging, for example, from 0.01% to 30% by weight relative to the total weight of the composition.

Non-limiting representatives of at least one surfactant may be chosen, for example, from:

anionic surfactants chosen, for example, from optionally unsaturated fatty acid salts having, for example, from 12 to 18 carbon atoms, alkali metal salts of salts of organic bases with (C₁₂-C₁₈) alkylsulfuric acids, alkali metal salts of salts of organic bases with (C₁₂-C₁₈) alkylsulfonic acids, alkali metal salts of salts of organic bases with (C₆-C₁₈) alkylarylsulfonic acids, and ether sulfates;

nonionic surfactants, chosen, for example, from polyalkoxylated surfactants and polyglycerolated surfactants, such as fatty acids, fatty acid amides, fatty alcohols, fatty alkylphenols, esters of fatty acids, esters of fatty polyols, alkanediols, alkyl ethers of alkanediols; and a least one compound chosen, for example, from alkyl carbamates of triglycerol, oxyethylenated derivatives of lanolin alcohols, propoxylated derivatives of lanolin alcohols, and lanolin fatty acids; and

cationic surfactants chosen, for example, from quaternary ammonium derivatives.

The at least one wax may further be combined with at least one fatty additive chosen, for example, from fatty oily additives, fatty pasty additives, fatty, oily and pasty additives, fat-soluble additives and fat-soluble active agents.

Non-limiting examples of at least one fatty additive may be chosen, for example, from vegetable oils such as sunflower oil, and jojoba oil; mineral oils such as paraffin oil; silicone oils; petroleum jelly; lanolin; fluorinated oils; hydrocarbon oils with at least one perfluorinated group; and esters of fatty alcohols.

It is possible to further introduce into the microparticulate waxy phase at least one fat-soluble additive and fat-soluble active ingredients chosen, for example, from UV-screening agents, fat-soluble vitamins, and fat-soluble cosmetic active agents.

C. The at Least One Film-forming Polymer

The composition according to the invention may comprise at least one film-forming polymer which may impart to the composition at least one good retention property chosen, for example, from resistance to water, such as for example, rain, tears, and bathing water, resistance to rubbing, and resistance to sebum.

The at least one film-forming polymer may be a polymer in a phase chosen from at least one aqueous phase or at least one liquid fatty phase, such as for example, a polymer solubilized or dispersed in the form of particles in the at least one aqueous phase of the composition, or a polymer solubilized or dispersed in the form of particles in the at least one liquid fatty phase. The composition may comprise a mixture of these polymers.

The at least one film-forming polymer may be present in the composition according to the invention in a dry matter content ranging, for example, from 0.1% to 60% by weight relative to the total weight of the composition, such as from 0.5% to 40% by weight, and further from 1 % to 30% by weight relative to the total weight of the composition.

For purposes of the invention, the expression “at least one film-forming polymer” is understood to mean at least one polymer capable of forming, on its own or in the presence of at least one film-forming aid, a continuous and adherent film on a support, for example, on keratinous materials.

The at least one film-forming polymer which may be used may be capable of forming a hydrophobic film, that is to say, the at least one polymer whose film has a water-solubility of less than 1% by weight at 25° C.

Non-limiting examples of the at least one film-forming polymer which can be used in the composition of the present invention may be chosen, for example, from synthetic polymers, such as free-radical polymers, and polycondensate polymers, and optionally modified polymers of natural origin.

The expression “free-radical film-forming polymer” is understood to mean a polymer obtained by polymerization of monomers, such as monomers with ethylenic unsaturation, each monomer being capable of homopolymerizing (in contrast to polycondensates).

Non-limiting examples of at least one film-forming polymer which are free-radical polymers may be chosen, for example, from vinyl polymers, and vinyl copolymers, such as acrylic polymers. The vinyl polymers may be formed, for example, from the polymerization of ethylenically unsaturated monomers having at least one acid group, esters of ethylenically unsaturated monomers having at least one acid group, and amides of ethylenically unsaturated monomers having at least one acid group.

Non-limiting representatives of monomers having at least one acid group, may be chosen, for example, from α,β-ethylenic unsaturated carboxylic acids such as acrylic acid, methacrylic acid, crotonic acid, maleic acid and itaconic acid. In one embodiment of the invention, the monomers having at least one acid group may be chosen, for example, from (meth)acrylic acid and crotonic acid.

Non-limiting examples of esters of monomers having at least one acid group may be chosen, for example, from esters of (meth)acrylic acid (also called (meth)acrylates), such as for example, alkyl(meth)acrylates such as (C₁-C₃₀)alkyl(meth)acrylates and (C₁-C₂₀) alkyl(meth)acrylates; aryl(meth)acrylates, such as (C₆-C₁₀)aryl(meth)acrylates; and hydroxyalkyl (meth)acrylates, such as, (C₂-C₆)hydroxyalkyl(meth)acrylates.

Among the alkyl (meth)acrylates, there may be mentioned those chosen, for example, from methyl methacrylate, ethyl methacrylate, butyl methacrylate, isobutyl methacrylate, 2-ethylhexyl methacrylate, lauryl methacrylate and cyclohexyl methacrylate.

Among the hydroxyalkyl (meth)acrylates, there may be mentioned those chosen, for example, from hydroxyethyl acrylate, 2-hydroxypropyl acrylate, hydroxyethyl methacrylate and 2-hydroxypropyl methacrylate.

Among the aryl (meth)acrylates, there may be mentioned those chosen, for example, from benzyl acrylate and phenyl acrylate.

In one embodiment of the invention, the esters of (meth)acrylic acid employed may be those chosen from alkyl (meth)acrylates.

According to the present invention, the alkyl group of the esters may optionally be fluorinated or perfluorinated, that is to say, some or all of the hydrogen atoms of the alkyl group may be substituted with fluorine atoms.

Among the non-limiting representatives of amides of the monomers having at least one acid group, there may be mentioned those chosen, for example, from (meth)acrylamides, such as N-alkyl(meth)acrylamides, chosen, fore example from N-(C₂-C₁₂)alkyl(meth)acrylamides. Among the N-alkyl(meth)acrylamides, there may be mentioned those chosen, for example, from N-ethylacrylamide, N-t-butylacrylamide, N-t-octylacrylamide and N-undecylacrylamide.

The at least one film-forming polymer may be vinyl polymers formed from the homopolymerization or copolymerization of monomers chosen, for example, from vinyl esters and styrene monomers. In another embodiment of the invention, the monomers chosen from vinyl esters and styrene monomers may also be polymerized with monomers chosen, for example, from monomers having at least one acid group, esters of monomers having at least one acid group, and amides of monomers having at least one acid group, such as those monomers previously mentioned above.

In one embodiment, the vinyl polymers are formed from the polymerization of monomers chosen from vinyl esters, styrene monomers, α,β-ethylenic unsaturated carboxylic acids, esters of α,β-ethylenic unsaturated carboxylic acids, and amides of α,β-ethylenic unsaturated carboxylic acids.

Non-limiting representatives of vinyl esters may be chosen, for example, from vinyl acetate, vinyl neodecanoate, vinyl pivalate, vinylbenzoate and vinyl t-butyl benzoate.

Non-limiting examples of styrene monomers may be chosen, for example, from styrene and alpha-methylstyrene.

The list of monomers which may be used to form the at least one film-forming monomer in accordance with the invention is not limiting and it is possible to use any monomer known to a person skilled in the art chosen, for example, from acrylic monomers and vinyl monomers (including the acrylic and vinyl monomers modified by at least one silicone chain).

Among the at least one film-forming polycondensates, there may be mentioned those chosen, for example, from polyurethanes, polyesters, polyester amides, polyamides, epoxy ester resins, and polyureas.

The polyurethanes may be chosen, for example, from anionic polyurethanes, cationic polyurethanes, nonionic polyurethanes, amphoteric polyurethanes, anionic polyurethane-acrylics, cationic polyurethane-acrylics, nonionic polyurethane-acrylics, amphoteric polyurethane-acrylics, anionic polyurethane-polyvinyl-pyrrolidones, cationic polyurethane-polyvinyl-pyrrolidones, nonionic polyurethane-polyvinyl-pyrrolidones, amphoteric polyurethane-polyvinyl-pyrrolidones, anionic polyester-polyurethanes, cationic polyester-polyurethanes, nonionic polyester-polyurethanes, amphoteric polyester-polyurethanes, anionic polyether-polyurethanes, cationic polyether-polyurethanes, nonionic polyether-polyurethanes, amphoteric polyether-polyurethanes, anionic polyureas, cationic polyureas, nonionic polyureas, amphoteric polyureas, anionic polyurea-polyurethanes, cationic polyurea-polyurethanes, nonionic polyurea-polyurethanes, and amphoteric polyurea-polyurethanes.

The polyesters may be obtained, in a known manner such as by polycondensation of dicarboxylic acids with polyols, for example, diols.

The dicarboxylic acids may be chosen, for example, from aliphatic dicarboxylic acids, alicyclic dicarboxylic acids, and aromatic dicarboxylic acids. Non-limiting examples of dicarboxylic acids may be chosen from oxalic acid, malonic acid, dimethylmalonic acid, succinic acid, glutaric acid, adipic acid, pimelic acid, 2,2-dimethylgutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalene-dicarboxylic acid, and 2,6-naphthalenedicarboxylic acid. In one embodiment of the invention, these dicarboxylic acid monomers may be used alone or in combination with at least two other dicarboxylic acid monomers.

In another embodiment, the dicarboxylic acid monomers are chosen from phthalic acid, isophthalic acid, and terephthalic acid.

The diols may be chosen, for example, from aliphatic diols, alicyclic diols, and aromatic diols. In one embodiment of the invention, the diols are chosen, for example, from ethylene glycol, diethylene glycol, triethylene glycol, 1,3-propanediol, cyclohexanedimethanol and 4-butanediol.

Other polyols which may be employed in forming the polyesters are those chosen, for example, from glycerol, pentaerythritol, sorbitol, and trimethylolpropane.

The polyester amides may be obtained in a manner similar to the polyesters, for example, by polycondensation of diacids with diamines or amino alcohols. As non-limiting representatives of diamines, there may be used those chosen, for example, from ethylenediamine, hexamethylenediamine, meta-phenylenediamine, and para-phenylenediamine. A non-limiting example of an aminoalcohol which may be used is monoethanolamine.

The polyesters used in accordance with the invention, may be formed with at least one monomer bearing at least one —SO₃M group, wherein M is a group chosen, for example, from a hydrogen atom, an ammonium ion (NH₄⁺) and a metal ion chosen, for example, from Na⁺, Li⁺, K⁺, Mg²⁺, Ca²⁺, Cu²⁺, Fe^{2 +}, and Fe³⁺ ions. In addition, bifunctional aromatic monomers having at least one —SO₃M group may also be used.

The aromatic ring of the bifunctional aromatic monomer carrying at least one —SO₃M group as described above may be chosen, for example, from benzene, naphthalene, anthracene, diphenyl, oxydiphenyl, sulphonyldiphenyl and methylenediphenyl rings.

Non-limiting examples of a bifunctional aromatic monomer carrying at least one —SO₃M group may be chosen, for example, from sulphoisophthalic acid, sulphoterephthalic acid, sulphophthalic acid, and 4-sulphonaphthalene-2,7-dicarboxylic acid.

In one embodiment of the invention, copolymers based on isophthalate/sulphoisophthalate, such as for example, copolymers obtained by condensation of diethylene glycol, cyclohexanedimethanol, isophthalic acid and sulphoisophthalic acid may be employed. Such polymers are sold, for example, under the trade name EASTMAN AQ by the company Eastman Chemical Products.

Non-limiting representatives of optionally modified polymers of natural origin may be chosen, for example, from shellac resin, sandarac gum, dammars, elemis, copals, and water-insoluble cellulosic polymers.

According to one embodiment of the composition according to the invention, the at least one film-forming polymer may be present in the form of particles in aqueous dispersion, generally known as latex or pseudolatex. The techniques for preparing these dispersions are well known to persons skilled in the art.

The composition in accordance with the invention may advantageously form a deposit on the keratinous materials which has at least one of the following properties: is resistant to cold water and can be removed as make-up with hot water. That is to say, hot water is understood to mean water heated to a temperature of at least 35° C., such as for example, water ranging from 35° C. to 50° C. The deposit can be easily removed with hot water, for example, hot water containing no detergent agent such as soap.

Among the non-limiting representatives of commercially available aqueous dispersions of at least one film-forming polymer, there may be mentioned those chosen, for example, from acrylic dispersions sold under the names such as NEOCRYL XK-90, NEOCRYL A-1070, NEOCRYL A-1090, NEOCRYL BT-62, NEOCRYL A-1079, and NEOCRYL A-523 by the company Avecia-Neoresins; DOW LATEX 432 by the company Dow Chemical; DAITOSOL 5000 AD by the company Daito Kasey Kogyo; and aqueous dispersions of polyurethane sold under the names NEOREZ R-981, and NEOREZ R-974 by the company Avecia-Neoresins; AVALURE UR-405, AVALURE UR-410, AVALURE UR-425, AVALURE UR-450, SANCURE 875, SANCURE 861, SANCURE 878, and SANCURE 2060 by the company Goodrich; IMPRANIL 85 by the company Bayer; and AQUAMERE H-1511 by the company Hydromer.

As aqueous dispersions of at least one film-forming polymer, there may also be mentioned the dispersions of polymers formed from free-radical polymerization of at least one free-radical monomer located inside and/or partially at the surface, of preexisting particles of at least one polymer chosen, for example, from polyurethanes, polyureas, polyesters, polyesteramides, and alkyds. These polymers are generally called hybrid polymers.

Another variant embodiment of the composition according to the invention is that the at least one film-forming polymer may be chosen from water-soluble polymers and is therefore present in the aqueous phase of the composition in solubilized form. Among the non-limiting representatives of water-soluble film-forming polymers, there may be mentioned those chosen, for example, from:

proteins chosen, for example, from proteins of plant origin, such as wheat proteins, and soya bean proteins, and proteins of animal origin such as keratin, for example keratin hydrolysates and sulfonic keratins;

polymers chosen, for example, from anionic, cationic, amphoteric, and nonionic polymers of chitins, and anionic, cationic, amphoteric, and nonionic polymers of chitosans;

cellulose polymers chosen, for example, from hydroxyethyl cellulose, hydroxypropyl cellulose, methyl cellulose, ethyl hydroxyethyl cellulose, carboxymethyl cellulose, and quaternized derivatives of cellulose;

acrylic polymers and acrylic copolymers, such as for example, polyacrylates and polymethacrylates;

vinyl polymers, chosen for example, from polyvinylpyrrolidones, polyvinyl alcohols, copolymers of methyl vinyl ether and maleic anhydride, copolymers of vinyl acetate and crotonic acid, copolymers of vinylpyrrolidone and vinyl acetate, and copolymers of vinylpyrrolidone and caprolactam; and

optionally modified polymers of natural origin chosen, for example, from gum arabic, guar gum, xanthan derivatives, karaya gum, alginates carrageenans, glycosaminoglycans shellac resin, sandarac gum, dammars, elemis, copals, deoxyribonucleic acid, mucopolysaccharides such as for example, hyaluronic acid, and chondroitin sulphate, and hyaluronic acid derivatives.

In one embodiment of the invention, the composition may further comprise at least one liquid fatty phase. In yet another variant embodiment of the composition according to the invention, the at least one film-forming polymer may be present in the at least one liquid fatty phase.

The at least one liquid fatty phase may comprise a volatile liquid fatty phase, optionally mixed with a nonvolatile liquid fatty phase.

For purposes of this invention, the expression “volatile liquid fatty phase” is understood to mean any nonaqueous medium capable of evaporating from the skin in less than one hour. This volatile liquid fatty phase comprises, for example, oils having a vapor pressure, at room temperature and at atmospheric pressure, ranging from 10⁻³ to 300 mmHg (0.13 Pa to 40,000 Pa). The liquid fatty phase may comprise any physiologically acceptable oil, for example, a cosmetically acceptable oil chosen, for example, from oils of mineral origin, oils of animal origin, oils of plant origin and oils of synthetic origin, carbonaceous oils, hydrocarbon oils, fluorinated oils, silicone oils. It should be noted that the mixtures of the above-mentioned oils should form a homogeneous and stable mixture and be compatible with the use envisaged.

The total liquid fatty phase of the composition (the nonvolatile liquid fatty phase and the volatile liquid fatty phase) according to the invention, may be present in an amount ranging, for example, from 5% to 98% by weight relative to the total weight of the composition, such as, from 20 to 85% by weight relative to the total weight of the composition.

The nonvolatile liquid fatty phase may be present in the composition according to the invention, in an amount ranging, for example, from 0 to 80% by weight relative to the total weight of the composition, such as from 0.1 to 80% by weight and further still, from 1 to 50% by weight relative to the total weight of the composition.

As compounds which may comprise the liquid fatty phase which can be used in the invention, there may thus be mentioned those compounds chosen, for example, from fatty acid esters; higher fatty acids; higher fatty alcohols; optionally phenylated polydimethylsiloxanes (PDMS), such as phenyltrimethicones; polydimethylsiloxanes (PDMS) optionally substituted with at least one group chosen, for example, from aliphatic groups, aromatic groups, fluorinated groups, and functional groups chosen, for example, from hydroxyl, thiol and amine groups; polysiloxanes modified with at least one group chosen from fatty acids, fatty alcohols, and polyoxyalkylenes; fluorinated silicones; and perfluorinated oils.

In one embodiment of the invention, at least one oil which is volatile at room temperature may be used. After evaporation of the at least one volatile at room temperature oil, a nonsticky, supple film-forming deposit may be obtained. These volatile oils may also facilitate the application of the composition to the keratinous materials.

Non-limiting representatives of the volatile oils may be chosen, for example, from hydrocarbon oils and silicone oils optionally containing at the end of a silicone or pendent chain at least one group chosen from alkyl groups and alkoxy groups.

Among the non-limiting representatives of volatile silicone oils which may be used in the composition of the invention, there may be mentioned those chosen, for example, from linear and cyclic silicones having 2 to 7 silicon atoms, wherein the silicones optionally comprise at least one group chosen from (C₁-C₁₀) alkyl groups and (C₁-C₁₀) alkoxy groups. Non-limiting examples of volatile silicone oils may be chosen, for example, from octamethylcyclotetrasiloxane, decamethylcyclopentasiloxane, hexadecamethylcyclohexasiloxane, heptamethylhexyltrisiloxane, and heptamethyloctyltrisiloxane.

As non-limiting representatives of volatile hydrocarbon oils, there may be mentioned those chosen, for example, from (C₈-C₁₆) isoparaffins such as ‘ISOPARs’, PERMETYLs and isododecane. In one embodiment of the invention, isododecane is the volatile hydrocarbon oil used.

These volatile oils may be present in the composition in accordance with the invention, in an amount ranging, for example, from 5 to 94.9% by weight relative to the total weight of the composition, and such as from 20 to 85% by weight relative to the total weight of the composition.

Non-limiting example of oils which may be used in the liquid fatty phase are disclosed in application EP-A-749747, the disclosure of which relating to oils which may comprise the liquid fatty phase is specifically incorporated by reference herein. As nonaqueous medium, there may also be used those described in the document FR-A-2 710 646 by L.V.M.H., the disclosure of which relating to nonaqueous medium is specifically incorporated by reference herein.

According to another variant embodiment of the composition according to the invention, the at least one film-forming polymer may be present in the form of surface-stabilized particles dispersed in the at least one liquid fatty phase.

The dispersion of surface-stabilized particles of polymer may be manufactured as described in the document EP-A-749747, the disclosure of which relating to the manufacture of surface-stabilized particles is specifically incorporated by reference herein. The dispersion of surface-stabilized particles may also be obtained by dispersion polymerization, that is to say, by precipitation of the polymer during formation. In this technique, the formed particles may be protected with at least one stabilizer.

The choice of the liquid fatty phase is made by a person skilled in the art according to the nature of the monomers constituting the polymer and the nature of the at least one stabilizer, as indicated below.

The polymer particles are surface-stabilized using at least one stabilizer which may be chosen, for example, from block polymers, graft polymers and random polymers.

Among the graft polymers, there may be mentioned those chosen, for example, from silicone polymers grafted with at least one hydrocarbon chain, and hydrocarbon polymers grafted with at least one silicone chain.

Other non-limiting representatives of suitable graft polymers are graft copolymers having, for example, an insoluble polyacrylic backbone with soluble grafts of the poly(12-hydroxystearic) acid type.

Other non-limiting examples of graft polymers may include those chosen, for example, from block and graft-block copolymers, wherein the block copolymers and graft-block copolymers comprise at least one polyorganosiloxane block type and at least one block of a free-radical polymer. In one embodiment, graft copolymers of the acrylic/silicone type may be used when, for example, the nonaqueous medium is silicone-containing.

The at least one stabilizer may also be chosen, for example, from block and graft-block copolymers comprising at least one block of polyorganosiloxane type and at least one polyether. The polyorganosiloxane block may be chosen, for example, from polydimethylsiloxanes and poly(C₂-C₁₈)alkylmethylsiloxanes; and the polyether block may be chosen, for example, from (C₂-C₁₈)polyalkylenes, such as for example, polyoxyethylene and polyoxypropylene. In one embodiment, the at least one stabilizer may be chosen from dimethicone copolyols and (C₂-C₁₈)alkyl methicone copolyols. It is possible to use, for example, the dimethicone copolyols sold under the name “DOW CORNING 3225C” by the company Dow Corning, or the lauryl methicone copolyol sold under the name “DOW CORNING Q2-5200” by the company Dow Corning.

As block or graft-block copolymers which may be used as the at least one stabilizer, there may be mentioned, for example, copolymers comprising at least one block formed from the polymerization of at least one ethylenic monomer with at least one optionally conjugated ethylenic bond such as for example, ethylene, butadiene, isoprene, and at least one block chosen from styrene polymers. When the ethylenic monomer comprises several optionally conjugated ethylenic bonds, the residual ethylenic unsaturations after the polymerization are generally hydrogenated. Thus, in a known manner, the polymerization of isoprene leads, after hydrogenation, to the formation of the ethylene/propylene block, and the polymerization of butadiene leads, after hydrogenation, to the formation of the ethylene-butylene block. Among these block copolymers, there may be mentioned “diblock” type copolymers and “triblock” type copolymers chosen, for example, from polystyrene/polyisoprene type copolymers and polystyrene/polybutadiene type copolymers such as those sold under the name “LUVITOL HSB” by BASF; polystyrene/copoly(ethylene-propylene) type copolymers, such as those sold under the name “KRATON” by Shell Chemical Co.; and polystyrene/copoly(ethylene-butylene) type copolymers.

Further non-limiting representatives of block or graft-block copolymers may include those copolymers comprising at least one block formed from the polymerization of at least one ethylenic monomer, such as ethylene, isobutylene, and at least one block of an acrylic polymer such as methyl methacrylate. There may be also mentioned as non-limiting examples those copolymers chosen, for example, from diblock poly(methyl methacrylate)/polyisobutylene copolymers, triblock poly(methyl methacrylate)/polyisobutylene copolymers, and graft copolymers having a poly(methyl methacrylate) backbone and at least one polyisobutylene graft.

Other non-limiting examples of block or graft-block copolymers are those copolymers chosen, for example from copolymers comprising at least one block formed from the polymerization of at least one ethylenic monomer and at least one block of a polyether, for example, (C₂-C₁₈)polyoxyalkylenes, such as, polyoxyethylene and polyoxypropylene, diblock polyoxyethylene/polybutadiene copolymers, diblock polyoxyethylene/polyisobutylene copolymers, triblock polyoxyethylene/polybutadiene copolymers, and triblock polyoxyethylene/polyisobutylene copolymers.

As at least one stabilizer, it is also possible to use copolymers of (C₁-C₄) alkyl (meth)acrylates and (C₈-C₃₀) alkyl (meth)acrylates. There may be mentioned those copolymers chosen, for example, from stearyl methacrylate/methyl methacrylate copolymers.

In one embodiment of the invention, the at least one stabilizer, is chosen from graft polymers and block polymers, so as to have better interfacial activity. For example, block polymers and graft polymers which are insoluble in the synthesis solvent may provide a more voluminous covering at the surface of the particles.

When the liquid fatty phase comprises at least one silicone oil, the at least one stabilizer may be chosen, for example, from block and graft-block copolymers comprising at least one polyorganosiloxane block and at least one free-radical polymer block chosen, for example, from polyethers and polyesters such as polyoxy(C₂-C₈)alkylenes, polyoxypropylenated block polymers, and oxyethylenated block polymers.

When the liquid fatty phase does not comprise a silicone oil, the at least one stabilizer may be chosen, for example, from:

(a) block copolymers and graft-block copolymers formed, for example, from at least one block chosen from polyorganosiloxane type polymers, and at least one block chosen from free-radial polymers, polyethers, and polyesters;

(b) copolymers formed, for example, from at least one first acrylate chosen, for example, from (C₁-C₄)alkyl acrylates and (C₁-C₄)alkyl methacrylates, and at least one second acrylate chosen, for example, from (C₈-C₃₀)alkyl acrylates and (C₈-C₃₀)alkyl methacrylates;

(c) block and graft-block copolymers formed, for example, from at least one block formed from polymerization of at least one ethylenic monomer having at least one conjugated ethylenic bond, and at least one block chosen, for example, from vinyl polymers, acrylic polymers, polyethers, and polyesters.

In one embodiment of the invention, diblock polymers are used as the at least one stabilizer.

Dispersions of at least one film-forming polymer in a liquid fatty phase, in the presence of stabilizers, are described in the documents: EP-A-749746, EP-A-923928, EP-A-930060, which are all incorporated by way of reference herein.

The size of the particles of the polymers in dispersion either in the aqueous phase or in the liquid fatty phase may range, for example, from 5 nm to 600 nm, such as, from 20 nm to 300 nm.

In yet another variant embodiment of the composition according to the invention, the at least one film-forming polymer may be solubilized in the liquid fatty phase. In this situation, the at least one film-forming polymer is then said to be at least one fat-soluble polymer. That is, the at least one film-forming polymer may be chosen from fat-soluble copolymers and fat-soluble homopolymers.

By way of example of fat soluble polymers, there may be mentioned a copolymer chosen from copolymers of formula (I):

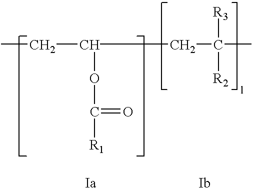

wherein:

R₁ is chosen from saturated, linear, and branched, hydrocarbon chains comprising from 1 to 19 carbon atoms;

R₂ is a group chosen from:

a) —O—CO—R₄, wherein R₄ is chosen from saturated, linear, and branched, hydrocarbon chains comprising from 1 to 19 carbon atoms provided that R₄ is different from R₁,

b) —CH₂—R₅, wherein R₅ is chosen from saturated, linear, and branched, hydrocarbon chains comprising from 5 to 25 carbon atoms,

c) —O—R₆, wherein R₆ is chosen from saturated hydrocarbon chains comprising from 2 to 18 carbon atoms, and

d) —CH₂—O—CO—R₇, wherein R₇ is chosen from saturated, linear, and branched, hydrocarbon chains comprising from 1 to 19 carbon atoms,

R₃ is a hydrogen group when R₂ is a group chosen from a), b), and c), and

R₃ is a methyl group when R₂ is a group d), provided that:

the copolymer of formula (I) is formed from at least 15% by weight relative to the total weight of the copolymer of at least one monomer having saturated and branched hydrocarbon chains comprising at least 7 carbon atoms, wherein said monomer is derived from a unit chosen from (Ia) and (Ib).

In one embodiment of the invention, the copolymers of formula (I) may be formed from the copolymerization of at least one vinyl ester (corresponding to the unit (Ia)) and of at least one other monomer (corresponding to the unit (Ib)) chosen, for example, from α-olefins, alkyl vinyl ethers, allyl esters and methallyl esters.

When, in the unit (Ib), R₂ is a group chosen from —CH₂—R₅, —O—R₆, and —CH₂—O—CO—R₇, as defined above, the copolymer of formula (I) may comprise 50 to 95 mol % of at least one unit (Ia) and 5 to 50 mol % of at least one unit (Ib).

In addition, the copolymers of formula (I) may be from the copolymerization of at least one first vinyl ester and at least one second vinyl ester, wherein the at least one second vinyl ester is different from the at least one first vinyl ester. In this case, the copolymers of formula (I) may comprise from 10 to 90 mol % of at least one unit (Ia) and from 10 to 90 mol % of at least one unit (lb), wherein R₂ is chosen from the group —O—CO—R₄.

Among the non-limiting representatives of vinyl esters leading to the unit of formula (Ia), or to the unit of formula (Ib), wherein R₂ is the group —O—CO—R₄, there may be mentioned those vinyl esters chosen, for example, from vinyl acetate, vinyl propionate, vinyl butanoate, vinyl octanoate, vinyl decanoate, vinyl laurate, vinyl stearate, vinyl isostearate, vinyl 2,2-dimethyloctanoate and vinyl dimethylpropionate.

Among the non-limiting representatives of the α-olefins leading to the unit of formula (Ib), wherein R₂ is the group —CH₂—R₅, there may be mentioned those α-olefins chosen, for example, from 1-octene, 1-dodecene, 1-octadecene, 1-eicosene and α-olefins comprising from 22 to 28 carbon atoms.

Among the non-limiting representatives of alkyl vinyl ethers leading to the unit of formula (Ib), wherein R₂ is the group —O—R₆, there may be mentioned those alkyl vinyl ethers chosen, for example, from ethyl vinyl ether, n-butyl vinyl ether, isobutyl vinyl ether, decyl vinyl ether, dodecyl vinyl ether, cetyl vinyl ether, and octadecyl vinyl ether.

Among the non-limiting representatives of allyl and methallyl esters leading to the unit of formula (lb), wherein R₂ is the group —CH₂—O—CO—R₇, there may be mentioned those esters chosen, for example, from allyl acetates, methallyl acetates, propionates, dimethylpropionates, butyrates, hexanoates, octanoates, decanoates, laurates, 2,2-dimethylpentanoates, stearates, and eicosanoates.

The copolymers of formula (I) may also be crosslinked using at least one crosslinking agent which may be intended to substantially increase the molecular weight of the copolymers of formula (I).

The crosslinking may be carried out during the copolymerization. In one embodiment of the invention, the at least one crosslinking agent may be chosen, for example, from vinyl crosslinking agents, allyl crosslinking agents, and methallyl crosslinking agents, such as for example, tetraallyloxyethane, divinylbenzene, divinyl octanedioate, divinyl dodecanedioate and divinyl octadecanedioate.

Non-limiting examples of copolymers of formula (I) which may be used in the composition according to the invention, may be copolymers chosen, for example, from vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyl-octanoate/vinyl laurate, allyl 2,2-dimethylpentanoate/vinyl laurate, vinyl dimethyl propionate/vinyl stearate, allyl dimethyl propionate/vinyl stearate, vinyl propionate/vinyl stearate crosslinked with 0.2% by weight of divinylbenzene relative to the total weight of monomers forming the copolymer, vinyl dimethyl propionate/vinyl laurate crosslinked with 0.2% by weight of divinylbenzene relative to the total weight of monomers forming the copolymer, vinyl acetate/octadecyl vinyl ether crosslinked with 0.2% by weight of tetraallyloxyethane relative to the total weight of monomers forming the copolymer, vinyl acetate/allyl stearate crosslinked with 0.2% by weight of divinylbenzene relative to the total weight of monomers forming the copolymer, vinyl acetate/1-octadecene crosslinked with 0.2% by weight of divinylbenzene relative to the total weight of monomers forming the copolymer, and allyl propionate/allyl stearate crosslinked with 0.2% by weight of divinylbenzene relative to the total weight of monomers forming the copolymer.

Non-limiting examples of fat-soluble film-forming polymers may be chosen from fat-soluble homopolymers formed, for example, from the homopolymerization of at least one monomer chosen, for example, from vinyl esters comprising 9 to 22 carbon atoms, (C₁₀-C₂₀) alkyl acrylates, and (C₁₀-C₂₀) alkyl methacrylates.

Non-limiting representatives of fat-soluble homopolymers may be chosen, for example, from polyvinyl stearate; polyvinyl stearate crosslinked with at least one compound chosen from divinylbenzene, diallyl ether, and diallyl phthalate; polystearyl (meth)acrylate optionally crosslinked with a compound chosen from ethylene glycol and tetraethylene glycol dimethacrylate; polyvinyl laurate optionally crosslinked with a compound chosen from ethylene glycol and tetraethylene glycol dimethacrylate; and polylauryl (meth)acrylate optionally crosslinked with a compound chosen from ethylene glycol and tetraethylene glycol dimethacrylate.

The fat-soluble copolymers and homopolymers defined above are known and described, for example, in application FR-A-2232303, the disclosure of which pertaining to fat-soluble copolymers and homopolymers is specifically incorporated by reference herein. The fat-soluble copolymers and homopolymers in accordance with the invention may have a weight-average molecular weight ranging, for example, from 2000 to 500,000, such as, from 4000 to 200,000.

Other non-limiting representatives of fat-soluble film-forming polymers which can be used in the invention, may be chosen, for example, from polyalkylenes for example, copolymers such as, polybutene; saturated and unsaturated, linear and branched, (C₁-C₈) alkyl celluloses such as ethyl cellulose and propyl cellulose; copolymers of vinylpyrrolidone (VP), for example, copolymers of vinylpyrrolidone and (C₂-C₄₀) alkenes, such as (C₃-C₂₀) alkenes. Non-limiting examples of VP copolymers which can be used in the invention, may be those chosen, for example, from VP/vinyl acetate, VP/ethyl methacrylate, butylated polyvinylpyrrolidone (PVP), VP/ethyl methacrylate/methacrylic acid, VP/eicoene, VP/hexadecene, VP/triacontene, VP/styrene and VP/acrylic acid/lauryl methacrylate.

The composition according to the invention may further comprise film-forming aids which may promote the formation of a film with the at least one film-forming polymer. The film-forming aids may be chosen from the compounds known to persons skilled in the art to be capable of fulfilling the desired function. Non-limiting representatives of film-forming aids may be chosen, for example, from plasticizing agents and coalescing agents.

D. The Additives

The composition in accordance with the invention may further comprise coloring matters chosen, for example, from pulverulent compounds and fat-soluble colorings. The coloring matters may be present in the composition in accordance with the invention in an amount, for example, ranging from 0.01 to 30% by weight relative to the total weight of the composition.

Non-limiting representatives of pulverulent compounds may be chosen, for example, from pigments and pearlescent agents, other than those described above, which are normally used in cosmetic or dermatological compositions. The pulverulent compounds may be present in the composition in accordance with the invention, in an amount ranging, for example, from 0.1 to 25% by weight relative to the total weight of the composition, such as, from 1 to 20% by weight relative to the total weight of the composition.

The pigments may be chosen from white and colored, inorganic and organic, pigments. Non-limiting examples of inorganic pigments may be chosen from titanium dioxide, optionally surface-treated zirconium oxides, optionally surface-treated cerium oxides, iron oxides, chromium oxides, manganese violet, ultramarine blue, chromium hydrate and ferric blue. Non-limiting examples of organic pigments may be chosen from carbon black, D & C type pigments, and lacquers based on units chosen, for example, from carmine, barium, strontium, calcium and aluminium.

The pearlescent pigments may be chosen, for example, from white pearlescent pigments such as mica coated with titanium, mica coated with bismuth oxychloride, colored pearlescent pigments such as for example, mica-titanium with iron oxides, mica-titanium with ferric blue, mica-titanium with chromium oxide, mica-titanium with an organic pigment of the above-mentioned type, and pearlescent pigments based, for example, on bismuth oxychloride.

The composition according to the invention may further comprise cosmetic agents commonly used in the cosmetic field, chosen, for example, from fillers, vitamins, trace elements, emollients, sequestrants, perfumes, dimethicone copolyols, ceramides, cohesion agents, alkalinizing agents, acidifying agents, surfactants, oils, moisturizing agents, perfumes and preservatives.

In one embodiment, the composition in accordance with the invention further comprises at least one additive chosen from film-forming aids, coloring matters, and cosmetic agents.

Of course, persons skilled in the art would be careful to choose these optional additional compounds, and the quantity of these optional additional compounds such that the advantageous properties of the composition according to the invention are not substantially, impaired by the addition envisaged.

The composition according to the invention may be prepared in a manner known to persons skilled in the art by the simple mixing of the optional additional compounds, and through incorporation of the microdispersion of at least one wax at room temperature or at a temperature, for example, ranging up to 45° C. after the mixing of the additives is complete. The fibers may be added after the microdispersion of at least one wax has been incorporated into the composition.

The composition according to the invention may be a care or treatment composition for the skin, chosen, for example, from products for concealing skin imperfections on keratinous materials, such as the face, the neck, the hands and the body, and a make-up composition chosen, for example, from a mascara, a foundation, a blusher, an eyeshadow, a product for the eyebrows, an eyeliner, a lip product, a nail varnish, and a make-up composition for the body, such as for example, a temporary tattoo composition and a semi-permanent tattoo composition.

One embodiment of the invention is a mascara product comprising:

a reservoir containing a mascara composition, in a physiologically acceptable medium comprising at least one aqueous phase, comprising at least one fiber and an aqueous microdispersion of particles of at least one wax; and

a system for applying the mascara composition onto a keratinous material, such as for example, eyelashes.

The reservoir may be provided, in a known manner, with an opening in which a draining system is lodged. The system for applying the mascara, may comprise a wand provided at a first end with a brush and at the second end with a cap intended to close the reservoir. Such a packaging system is illustrated in FIG. 7 of application EP-A-611170 the disclosure of which pertaining to the packaging system is specifically incorporated by way of reference herein.

Unless otherwise indicated, all numbers expressing quantities of ingredients, properties such as molecular weight, reaction conditions, and so forth used in the specification and claims are to be understood as being modified in all instances by the term “about”. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Any numerical value inherently contains certain errors necessarily resulting from the standard deviation found in their respective testing measurements.

The following examples are intended to illustrate the invention without in anyway limiting the scope thereof.

### EXAMPLE 1

A microdispersion of carnauba wax having the following composition was prepared:

| | | | |
|---|---|---|---|
| Carnauba wax | | 27 | g |
| Polyoxyethylenated (30 EO) glyceryl monostearate | | 6.75 | g |
| (TAGAT S from GOLDSCHMIDT) | | | |
| Ethanol | | 10 | g |
| Water | qs | 100 | g. |

The wax and the surfactant were heated to 90° C. while the mixture was homogenized by moderate stirring. While stirring continued, water, heated to 90° C., was incorporated in the mixture. The composition was cooled to room temperature and ethanol was added in order to obtain a wax microdispersion having a mean particle diameter of 170 nm.

### EXAMPLE 2

A mascara having the following composition was prepared:

| | | | |
|---|---|---|---|
| Wax microdispersion of Example 1 | | 36 | g |
| Sulphopolyester (AQ 55S from the company | | 22 | g |
| Eastman Kodak) | | | |
| Polyamide fibers (3 mm long and 0.9 Dtex from | | 0.5 | g |
| the company Paul Bonte) | | | |
| Hydroxyethy/cellulose | | 0.6 | g |
| Propylene glycol | | 3 | g |
| Black iron oxide | | 5 | g |
| Preservatives | qs | | |
| Water | qs | 100 | g. |

The composition applied dried rapidly and adhered very well to the eyelashes and visibly lengthened the eyelashes. The make-up exhibited good retention in a humid atmosphere (30° C. at 80% relative humidity) and was easily removed by simple application of cotton wool impregnated with warm water. The mascara was cleanly removed in the form of a coating without leaving traces or a halo on the eyelids.

### EXAMPLE 3

A mascara having the following composition was prepared:

| | | | |
|---|---|---|---|
| Carnauba wax | | 5.5 | g |
| Rice bran wax | | 2.6 | g |
| Paraffin | | 2.6 | g |
| Beeswax | | 9.7 | g |
| Wax microdispersion of Example 1 | | 6.7 | g |
| Polyamide fibers (3 mm long and 0.9 Dtex | | 1 | g |
| from the company Paul Bonte) | | | |
| Talc | | 1 | g |
| Bentonite | | 5.5 | g |
| Vinyl acetate/allyl stearate copolymer (65/35) | | 7.4 | g |
| Polyvinyl laurate (Mexomer PP from CHIMEX) | | 0.7 | g |
| Isododecane | | 50.3 | g |
| Propylene carbonate | | 1.7 | g |
| D-Panthenol | | 0.2 | g |
| Pigments | | 4.9 | g |
| Preservatives | qs | | |
| Water | qs | 100 | g. |

The mascara was easily applied to the eyelashes and formed a make-up which lengthened and adhered well to the eyelashes.

What is claimed is:

1. A composition comprising, in a physiologically acceptable medium comprising at least one aqueous phase, at least one fiber and an aqueous microdispersion of particles of at least one wax.

2. A composition according to claim 1, wherein said at least one fiber is chosen from silk fibers, cotton fibers, wool fibers, flax fibers, cellulose fibers, polyamide fibers, rayon fibers, viscose fibers, acetate fibers, poly(p-phenylene-terephthalamide) fibers, acrylic polymer fibers, polyolefin fibers, glass fibers, silica fibers, carbon fibers, polytetrafluoroethylene fibers, insoluble collagen fibers, polyester fibers, polyvinyl chloride fibers, polyvinylidene chloride fibers, polyvinyl alcohol fibers, polyacrylonitrile fibers, chitosan fibers, polyurethane fibers, and polyethylene phthalate fibers.

3. A composition according to claim 1, wherein said at least one fiber comprises at least two polymers chosen from silks, cottons, wools, flaxes, celluloses, polyamides, rayons, viscoses, acetates, poly(p-phenylene-terephthalamide), acrylic polymers, polyolefins, polytetrafluoroethylenes, insoluble collagens, polyesters, polyvinyl chlorides, polyvinylidene chlorides, polyvinyl alcohols, polyacrylonitriles, chitosans, polyurethanes, and polyethylene phthalates.

4. A composition according to claim 1, wherein said at least one fiber is of synthetic origin.

5. A composition according to claim 1, wherein said at least one fiber is chosen from polyamide fibers, poly(p-phenylene-terephthalamide) fibers, cellulose fibers, and polyethylene fibers.

6. A composition according to claim 4, wherein said at least one fiber is chosen from resorbable synthetic fibers.

7. A composition according to claim 1, wherein said at least one fiber is chosen from polyterephthalic ester fibers and stainless steel wires.

8. A composition according to claim 1, wherein said at least one fiber has a length L, a diameter D, and the ratio L/D ranges from 3.5:1 to 2500:1.

9. A composition according to claim 8, wherein the ratio L/D ranges from 5:1 to 500:1.

10. A composition according to claim 9, wherein the ratio L/D ranges from 5:1 to 150:1.

11. A composition according to claim 8, wherein said length L ranges from 1 μm to 10 mm.

12. A composition according to claim 11, wherein said length L ranges from 0.1 mm to 5 mm.

13. A composition according to claim 12, wherein said length L ranges from 1 mm to 3.5 mm.

14. A composition according to claim 1, wherein said at least one fiber has a mean diameter ranging from 2 nm to 500 μm.

15. A composition according to claim 14, wherein said at least one fiber has a mean diameter ranging from 100 nm to 100 μm.

16. A composition according to claim 15, wherein said at least one fiber has a mean diameter ranging from 1 μm to 50 μm.

17. A composition according to claim 1, wherein said at least one fiber has a titre ranging from 0.15 to 30 denier.

18. A composition according to claim 17, wherein said at least one fiber has a titre ranging from 0.18 to 18 denier.

19. A composition according to claim 1, wherein said at least one fiber is present in an amount ranging from 0.01% to 10% by weight relative to the total weight of said composition.

20. A composition according to claim 19, wherein said at least one fiber is present in an amount ranging from 0.1% to 5% by weight relative to the total weight of said composition.

21. A composition according to claim 20, wherein said at least one fiber is present in an amount ranging from 0.3% to 2% by weight relative to the total weight of said composition.

22. A composition according to claim 1, wherein the particles of at least one wax have a mean particle size of less than 1 μm.

23. A composition according to claim 22, wherein the particles of at least one wax have a mean particle size of less than 0.5 μm.

24. A composition according to claim 1, wherein said at least one wax has a melting point ranging from 30° C. to 120° C.

25. A composition according to claim 1, wherein said at least one wax has a hardness ranging from 0.05 MPa to 15 MPa.

26. A composition according to claim 1, wherein said at least one wax has a hardness ranging from 6 MPa to 15 MPa.

27. A composition according to claim 1, wherein said at least one wax is chosen from beeswax, lanolin wax, Chinese waxes, rice wax, Carnauba wax, candelilla wax, ouricury wax, Esparto wax, cork fiber wax, sugarcane wax, Japan wax, sumac wax, montan wax, microcrystalline waxes, paraffins, ozokerite, polyethylene waxes, waxes obtained by Fisher-Tropsch synthesis, waxy copolymers and esters of waxes.

28. A composition according claim 1, wherein said at least one wax is present in a dry matter content ranging from 0.1% to 50% by weight relative to the total weight of the composition.

29. A composition according claim 28, wherein said at least one wax is present in a dry matter content ranging from 0.5% to 30% by weight relative to the total weight of the composition.

30. A composition according claim 1, wherein said at least one wax is present in a dry matter content ranging from 1% to 20% by weight relative to the total weight of the composition.

31. A composition according to claim 1, wherein said particles of said at least one wax further comprise at least one fatty additive chosen from fatty oily additives, fatty pasty additives, fat-soluble additives and fat-soluble active agents.

32. A composition according to claim 1, further comprising at least one surfactant.

33. A composition according to claim 1, further comprising at least one film-forming polymer.

34. A composition according to claim 33, wherein said at least one film-forming polymer is chosen from free-radical polymers, polycondensates, and optionally modified polymers of natural origin.

35. A composition according to claims 34, wherein said free-radical polymers are chosen from vinyl polymers formed from the polymerization of monomers chosen from monomers having at least one acid group, esters of monomers having at least one acid group, and amides of monomers having at least one acid group.

36. A composition according to claims 33, wherein said at least one film-forming polymer is chosen from vinyl polymers formed from the polymerization of monomers chosen from vinyl esters, styrene monomers, monomers having at least one acid group, esters of monomers having at least one acid group, and amides of monomers having at least one acid group.

37. A composition according to claims 36, wherein said at least one film-forming polymer is chosen from vinyl polymers formed from the polymerization of monomers chosen from α,β-ethylenic unsaturated carboxylic acids, esters of said α,β-ethylenic unsaturated carboxylic acids, amides of said α,β-ethylenic unsaturated carboxylic acids, vinyl esters, and styrene monomers.

38. A composition according to claim 34, wherein said polycondensates are chosen from polyurethanes, polyesters, polyester amides, polyamides, epoxy ester resins, and polyureas.

39. A composition according to claim 34, wherein said optionally modified polymers of natural origin are chosen from shellac resin, sandarac gum, dammars, elemis, copals, and water-insoluble cellulosic polymers.

40. A composition according to claim 1, wherein said at least one film-forming polymer is present in a dry matter content ranging from 0.1% to 60% by weight relative to the total weight of the composition.

41. A composition according to claim 40, wherein said at least one film-forming polymer is present in a dry matter content ranging from 0.5% to 40% by weight relative to the total weight of the composition.

42. A composition according to claim 41, wherein said at least one film-forming polymer is present in a dry matter content ranging from 1% to 30% by weight relative to the total weight of the composition.

43. A composition according to claim 33, wherein said at least one film-forming polymer is solubilized in said at least one aqueous phase or is dispersed in the form of particles in said at least one aqueous phase.

44. A composition according to claim 33, wherein said at least one film-forming polymer is a water-soluble polymer chosen from:
proteins chosen from proteins of plant origin and proteins of animal origin;
polymers chosen from anionic, cationic, amphoteric, and nonionic polymers of chitins; and anionic, cationic, amphoteric, and nonionic polymers of chitosans;
cellulose polymers chosen from hydroxyethyl cellulose, hydroxypropyl cellulose, methyl cellulose, ethyl hydroxyethyl cellulose, carboxymethyl cellulose, and quaternized derivatives of cellulose;
acrylic polymers, acrylic copolymers;
vinyl polymers; and
optionally modified polymers of natural origin chosen from gum arabic, guar gum, xanthan derivatives, karaya gum, alginates carrageenans, glycosaminoglycans, shellac resin, sandarac gum, dammars, elemis, copals, deoxyribonucleic acid, mucopolysaccharides chosen from hyaluronic acid, and chondroitin sulphate, and hyaluronic acid derivatives.

45. A composition according to claim 33, further comprising at least one liquid fatty phase.

46. A composition according to claim 45, wherein said at least one film-forming polymer is present in at least one phase chosen from said at least one aqueous phase and at least one liquid fatty phase.

47. A composition according to claim 45, wherein said at least one film-forming polymer is solubilized in said at least one liquid fatty phase or said at least one film-forming polymer is dispersed in the form of particles in said at least one liquid fatty phase.

48. A composition according to claim 47, wherein said at least one film-forming polymer dispersed in the form of particles in said at least one liquid fatty phase is surface-stabilized with at least one stabilizer.

49. A composition according to claim 47, wherein said at least one film-forming polymer is solubilized in said at least one liquid fatty phase.

50. A composition according to claim 49, wherein said at least one film-forming polymer is chosen from fat-soluble polymers.

51. A composition according to claim 50, wherein said fat-soluble polymers are chosen from:
a) fat-soluble homopolymers; and
b) fat-soluble copolymers of formula (I): 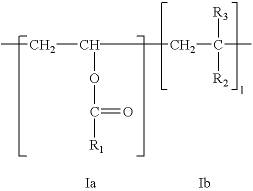
wherein:
R₁ is chosen from saturated, linear, and branched, hydrocarbon chains comprising from 1 to 19 carbon atoms;
R₂ is a group chosen from:
a) —O—CO—R₄, wherein R₄ is chosen from saturated, linear, and branched, hydrocarbon chains comprising from 1 to 19 carbon atoms provided that R₄ is different from R₁,
b) —CH₂—R₅, wherein R₅ is chosen from saturated, linear, and branched, hydrocarbon chains comprising from 5 to 25 carbon atoms,
c) —O—R₆, wherein R₆ is chosen from saturated hydrocarbon chains comprising from 2 to 18 carbon atoms, and
d) —CH₂—O—CO—R₇, wherein R₇ is chosen from saturated, linear, and branched, hydrocarbon chains comprising from 1 to 19 carbon atoms,
R₃ is a hydrogen group when R₂ is a group chosen from a), b), and c), and
R₃ is a methyl group when R₂ is a group d), provided that:
the copolymer of formula (I) is formed from at least 15% by weight relative to the total weight of the copolymer of at least one monomer having saturated and branched hydrocarbon chains comprising at least 7 carbon atoms, wherein said monomer is derived from a unit chosen from (Ia) and (Ib).

52. A composition according to claim 51, wherein said fat-soluble homopolymers are chosen from homopolymers formed from the homopolymerization of at least one monomer chosen from vinyl esters comprising 9 to 22 carbon atoms, (C₁₀-C₂₀) alkyl acrylates, and (C₁₀-C₂₀) alkyl methacrylates.

53. A composition according to claim 50, wherein said fat-soluble polymers have weight-average molecular weights ranging from 2000 to 500,000.

54. A composition according to claim 53, wherein said fat-soluble polymers have weight-average molecular weights ranging from 4000 to 200,000.

55. A composition according to claim 45, wherein said at least one liquid fatty phase comprises a volatile liquid fatty phase and a nonvolatile liquid fatty phase.

56. A composition according to claim 55, wherein said volatile liquid fatty phase is present in an amount ranging from 5% to 98% by weight relative to the total weight of said composition.

57. A composition according to claim 56, wherein said nonvolatile liquid fatty phase is present in an amount ranging from 0% to 80% by weight relative to the total weight of said composition.

58. A composition according to claim 1, further comprising at least one additive chosen from film-forming aids, coloring matters, and cosmetic agents.

59. A composition according to claim 58, wherein said at least one additive is chosen from thickeners, plasticizing agents, coalescing agents, fillers, waxes, surfactants, preservatives, oils, moisturizing agents, and perfumes.

60. A mascara, in a physiologically acceptable medium comprising at least one aqueous phase, comprising at least one fiber and an aqueous microdispersion of particles of at least one wax.

61. A mascara product comprising:
a reservoir containing a mascara composition comprising, in a physiologically acceptable medium comprising at least one aqueous phase, at least one fiber and an aqueous microdispersion of particles of at least one wax; and
a system for applying the mascara composition onto a keratinous material.

62. A mascara product according to claim 61, wherein said keratinous material is eyelashes.

63. A cosmetic method for make-up or care of a keratinous material comprising applying to the keratinous material a composition comprising, in a physiologically acceptable medium comprising at least one aqueous phase, comprising at least one fiber and an aqueous microdispersion of particles of at least one wax.

64. A cosmetic method according to claim 62, wherein said keratinous material is of human origin.

65. A method for covering eyelashes comprising applying to the eyelashes a composition comprising, in a physiologically acceptable medium comprising at least one aqueous phase, at least one fiber and an aqueous microdispersion of particles of at least one wax.

66. A method for obtaining a deposit adhering to a keratinous material comprising applying, to the keratinous material, a composition comprising, in a physiologically acceptable medium comprising at least one aqueous phase, at least one fiber and an aqueous microdispersion of particles of at least one wax to obtain the deposit which adheres to the keratinous material.

67. A method according to claim 66, further comprising at least one film-forming polymer.

68. A method according to claim 66, wherein said deposit which adheres to the keratinous material has at least one property chosen from resistance to cold water and removability as make-up with hot water.

69. A method according to claim 66, wherein the keratinous material is of human origin.
